(19) United States
(12) Patent Application Publication
(10) Pub. No.: US 2007/0055160 A1
(43) Pub. Date: Mar. 8, 2007

(54) Ultrasonic multiple beam transmission using single crystal transducer

(75) Inventor: Gary Ng, Bothell, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: Koninklijke Philips Electronics N.V., Eindhoven (NL)

(21) Appl. No.: 10/576,998

(22) PCT Filed: Nov. 1, 2004
(86) PCT No.: PCT/IB04/03569

§ 371 Date: Apr. 25, 2006

Related U.S. Application Data
(60) Provisional application No. 60/517,151, filed on Nov. 3, 2003.

(51) Int. Cl. A61B 8/00

(52) U.S. Cl. 600/447

(57) ABSTRACT

An ultrasonic imaging system uses a wide bandwidth transducer to transmit multiple simultaneous beams. The beams occupy different frequency bands of the transducer bandwidth and are steered in different beam directions. The received beams are separated by bandpass filters tuned to the different frequency bands. If the different frequency bands overlap, cross-talk between the two beams may be reduced by using coded pulses for the transmit beams and matched filters to separate the received echo signals of the simultaneous beams. a single crystal transducer is used as the wide bandwidth transducer.

This invention relates to ultrasonic diagnostic imaging and, more particularly, to ultrasonic imaging systems capable of transmitting multiple simultaneous beams.

Ultrasonic diagnostic imaging systems are often preferred for medical diagnoses of organs such as the heart due to their ability to perform real time imaging. The real time capability enables ultrasound to capture the movement of the beating heart and its valves, for instance. Blood flow can also be visualized in real time with ultrasound. To capture the motion of organs which are moving very rapidly such as a pediatric heart it is desirable to have a high frame rate which can image the motion smoothly. However, a limitation impeding high frame rates is the time required for a transmitted ultrasound wave to travel the required depth in the body and for the resultant echoes to return to the transducer. Since such a transmit-receive cycle is necessary when scanning each line used to produce an image, the number of lines required for an image frame and the time required to gather the echoes for each line, generally a function of the desired image depth, can impose a limit on the frame rate of display. Several transmit and receive techniques have been developed in an attempt to overcome this limitation. On the receive side, the reception of multiple lines from a single transmit beam will increase the frame rate, but can introduce artifacts related to the relation of each receive beam to the transmit beam center and can exhibit loss of spatial resolution. Display lines can be produced artificially by interpolating display lines between actual received lines. On the transmit side, attempts have been made to transmit multiple beams simultaneously. A difficulty with simultaneous beam transmission is that the echoes from the multiple transmit beams are being received by the transducer simultaneously and must be clearly segmented or separated after reception. Efforts for dealing with this problem of cross-talk between multiple beams are described in the paper “Golay Codes For Simultaneous Multi-mode Operation In Phased Arrays,” by B. B. Lee and E. S. Furgason, published in the Proceedings of the 1982 Ultrasonics Symposium at page 821 et seq., and in U.S. Pat. Nos. 5,276,654 and 6,221,022. These publications suggest different coding schemes or aperture configurations for each beam and transmitting the simultaneous beams at different focal regions. While these approaches improve the problem, the degree of separation of the echoes from each beam remains less than satisfactory. Accordingly it is desirable to augment or supplement these approaches with other solutions to the echo separation problem.

In accordance with the principles of the present invention, multiple beams are transmitted simultaneously using different frequency bands of a wide bandwidth transducer. In a preferred embodiment the wide bandwidth transducer is a single crystal transducer. The beams transmitted using the different frequency bands can be encoded so that the different codes can be separately distinguished upon reception. The use of the different frequency bands can cause the coding scheme to be more nearly orthogonal and hence the different echoes from the multiple beams can be more fully separately distinguishable due to frequency division. By transmitting multiple beams at the same time, fewer transmit-receive cycles are needed to scan a given volume or area, and the frame rate of display can be improved.

### IN THE DRAWINGS

FIG. 1 illustrates different frequency bands of a conventional transducer.

FIG. 2 illustrates another approach for obtaining different frequency bands by means of a conventional transducer.

FIG. 3 illustrates different frequency bands of a transducer constructed in accordance with the principles of the present invention.

FIG. 4 illustrates in block diagram form an ultrasonic imaging system constructed in accordance with the principles of the present invention.

FIG. 5 illustrates the filters of FIG. 4 in greater detail.

FIG. 6 illustrates the reception of a coded echo signal using a matched filter.

FIGS. 7a and 7b illustrate bandwidth and phase characteristics of a matched filter system.

FIGS. 8a and 8b illustrate bandwidth and phase characteristics of a mismatched filter system.

FIG. 9 illustrates the reception of a coded echo and subsequent compression of a coded echo.

FIGS. 10a-10c illustrate the benefit realized from the use of different Golay codes in a multi-pulse system.

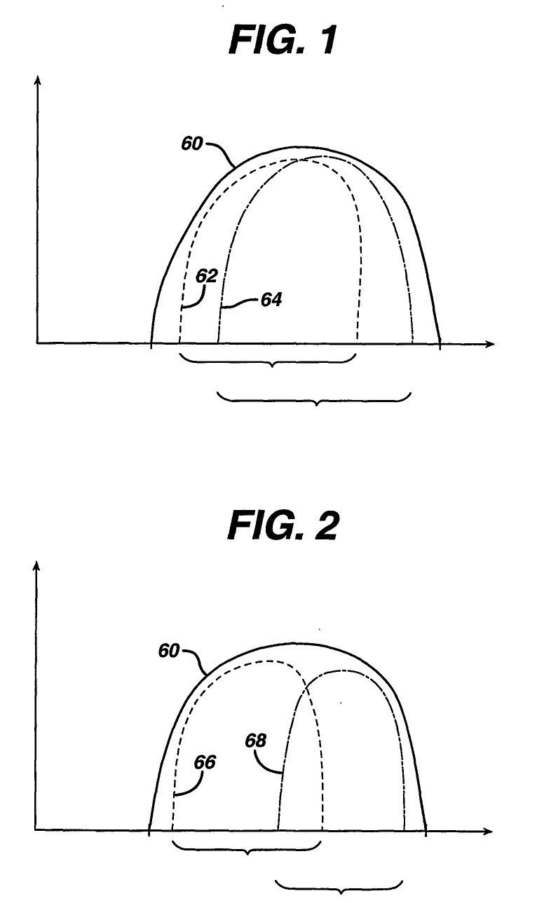

Referring first to FIG. 1, the passband 60 of a conventional PZT piezoelectric ultrasound transducer is shown. In this example the passband is shown extending from 2 to 5 MHz. When a transducer such as one of this conventional design is to transmit two beams simultaneously it is desirable to frequency encode the beams using different frequency bands for the different beams so that the resultant echoes can be distinguished by their different receive frequencies. However it is also desirable for the transmit bandwidth of each beam to be broad so that the resultant received beams exhibit good axial resolution. Thus, two different passbands 62 and 64 are used for the two different beams. While each passband is desirably wide so as to afford good axial resolution, it is also seen that the bands A and B of each of the passbands overlap each other to a considerable extent. This overlapping of the passbands can cause the received echoes to exhibit considerable cross-talk, where the echoes received from one beam in one transmit direction will contain components from other transmit beams transmitted simultaneously in other directions.

One way to improve the cross-talk problem is to use passbands 66 and 68 as shown in FIG. 2, where it is seen that bands A and B overlap only slightly in the center of the transducer passband 60. While this reduces the cross-talk problem, it also results in a narrowing of the band of each transmit beam. This undesirably degrades the axial resolution of the received echo signals.

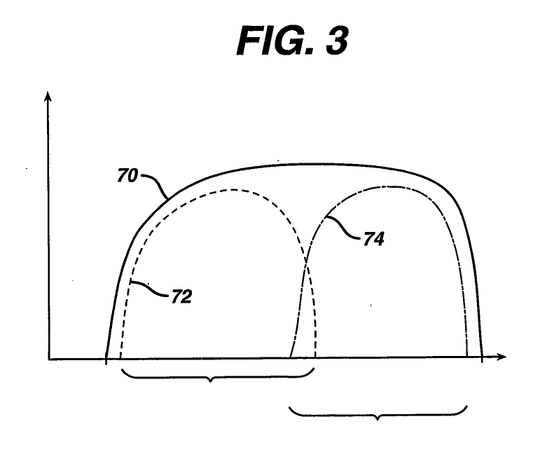

A solution to both problems in accordance with the present invention is shown in FIG. 3. This is to use a transducer with a wide passband 70. In this example the passband 70 is shown extending from 1.5 MHz to 6.5 MHz. This broad passband 70 can be used by separate transmit beam passbands 72 and 74, each of which exhibits a relatively broad bandwidth for good axial resolution. The central overlap area of the two bands A and B is relatively small.

A preferred transducer to use for the multi-beam wide passband transducer is one that is made by a single crystal fabrication process. Examples of single crystal transducers are those which are composed of PMN-PT and/or PZN-PT. For the purposes of the present invention, the term single crystal is used to denote oriented polycrystals in which the crystal comprises very few grains (all aligned in the same direction), and single grain crystals in which the crystal comprises only a single grain of material. To fabricate these elements, chemical grade PbO, MgO, ZnO, Nb₂O₅, and TiO₂ may be used to form PMN-PT and PZN-PT compositions. Once the compositions are formed, PMN-PT and PZN-PT single crystals may be grown using the Bridgman and flux technique, and may be oriented via the Laue back reflection method. Next, the crystals may be sliced using an inter-dimensional (ID) saw parallel to the (001), (011), and (111) planes to approximately 1 mm in thickness.

From Table I, it can be appreciated that several different thickness/width cut orientations can be beneficially used in creating a wideband transducer. Due to the particularly desirable properties obtained from single crystal wafers having <001> and <011> thickness orientations, these wafers represent the preferred orientations for crystals that may be used in constructing transducers. Once sliced, the wafers may then be lapped and polished. Gold coating may be applied to both surfaces of the wafers to form electrodes. The single crystal wafers may then be diced on a dicing saw into thin slivers with various width orientation cuts. The slivers may then be poled and measured at room temperature.

After completing transducer material fabrication, the electromechanical properties of the various single crystal slivers may be evaluated. Table I lists the piezoelectric and dielectric properties for various slivers. As shown in the table, very high effective coupling constants may be obtained for slivers k₃₃′=84% to 90%) constructed in accordance with the above description.

TABLE I
Effective Coupling Constants and Dielectric Constants
of PMN-PT and PZN-PT Slivers

| | Effective Coupling | Clamped Dielectric |
|---|---|---|
| | Constant (k₃₃) | Constant (K) |
| PMN-PT 30-32% | | |
| (rhombohedral) | | |
| <001>ₜ/<010>_{w} | 0.86-0.89 | 1400 |
| <011>ₜ/<211>_{w} | 0.90 | 1100 |
| <011>ₜ/<522>_{w} | 0.90 | 1100 |
| <011>ₜ/<311>_{w} | 0.90 | 1100 |
| <011>ₜ/<110>_{w} 35 degrees | 0.72 | 1100 |
| PZN-PT 4.5% PT | | |
| (rhombohedral) | | |
| <001>ₜ/<010>_{w} | 0.84-0.87 | 1100 |
| PZN-PT 8% PT | | |
| (rhombohedral) | | |
| <001>ₜ/<010>_{w} | 0.85-0.88 | 900 |

For one-dimensional (1D) transducer applications, the single crystal elements may be diced into one-dimensional or quasi-one dimensional sliver shapes where the length>height>width. Not only the thickness orientations, but also the width orientations affect the electromechanical properties of the slivers. As illustrated in Table I, the effective coupling constant (k₃₃′ for slivers) replaces the longitudinal coupling constant (k₃₃ for bars) due to the clamping effect from the length dimension of the sliver. By effectively selecting the thickness and width orientations, very high k₃₃′ (from 0.70 to 0.90) for sliver samples can be obtained, which is very close to the k₃₃ value of bar samples.

Utilizing the large coupling constant k₃₃ obtainable with such single crystals of PMN-PT and PZN-PT, in conjunction with additional improvements such as multiple matching layers, voltage biasing, and multiple-layer design, single crystal transducers can be designed with extremely wide bandwidth. In particular, the additional bandwidth achieved through the use of single crystal transducers provides a total bandwidth which can be separated into different passbands for multiply transmitted transmit beams. As will be understood by persons having ordinary skill in the art, this additional bandwidth creates several application possibilities which either were not possible with conventional transducers, or which were not nearly as useful due to the limitations of such transducers.

One disadvantage related to the use of PMN-PT and PZN-PT single crystals in manufacturing ultrasonic transducers concerns difficulty associated with acoustic matching. The problem of acoustic matching can, however, be overcome through the use of matching layers. In particular, the utilization of multiple matching layers can effectively couple the acoustic energy from the transducer into the body, therefore improving the bandwidth significantly.

In this regard, an ultrasonic transducer comprising single crystal element slivers of these materials may also includes multiple matching layers. A typical single crystal transducer may comprise a backing and an acoustic lens. Interposed between the single crystal slivers and the acoustic lens are, for example, three matching layers. The use of three such matching layers in combination with single crystal slivers render unexpectedly advantageous results in wideband ultrasonic transducer properties.

Table II illustrates modeled bandwidth data of PMN-PT single crystal transducers (<001>✓<010>_{w} or <011>✓<110>_{w 50-75} degree cuts) with various numbers of matching layers. As shown in Table II, approximately 105% of a −6 dB bandwidth was determined to be possible by using three matching layers.

TABLE II
Statistic Bandwidth Data of Modeled PMN-PT Single
Crystal Transducers with Multiple Acoustic Matching Layers

| | Bandwidth | | |
|---|---|---|---|
| | (−6 dB) | (−20 dB) | (−40 dB) |
| 2 Layer Design: | 95% | 120% | 160% |
| 3 Layer Design: | 105% | 130% | 160% |
| 4 Layer Design: | 113% | 135% | 165% |

A typical wideband phased-array transducer was built with 80 active elements with an element pitch of 254 μm. A single layer of PMN-PT single crystal (<001>✓<010>_{w}, and <011>✓<110>_{w 50-75 degrees} cuts) was used as the piezoelectric layer in conjunction with three matching layers to improve acoustic impedance matching. A room-temperature vulcanized (RTV) acoustic lens was added in front of the matching layers to obtain the acoustic focus. The transducer was integrated to an ultrasound imaging system as described below by way of a series inductor and a cable 6 feet in length.

The PMN-31% PT with sliver orientation of <001>✓<010>_{w} was used to build the transducer. The effective coupling constant (k₃₃′) of the sliver was 0.88 and clamping dielectric constant, K, was 1,200. The PMN-PT single crystal plate (<001> orientation) and matching layers were bonded together with epoxy and diced into a one-dimensional array. The thickness to width aspect ratio (t/w) of the sliver was about 0.5. More than 99% of the elements survived the transducer build. In the experiment, the center frequency was 2.7 MHz with −6 dB band edges of 1.15 MHz at the low frequency side (low corner frequency) and 4.1 MHz at the high frequency side (high corner frequency). As a result, the total −6 dB bandwidth for the transducer may be calculated as shown below.

$$\% BW = 100 * ( \frac{{UpperCorner}_{f} - {LoowerCorner}_{f}}{{Center}_{f}} )$$

$$\% BW = 100 * ( ( 4 / 1 - 1 / 15 ) / 2.7 ) = 109 \%$$

The −20 dB bandwidth was 130% for this transducer. The above data indicates that a very wide bandwidth (more than 100% of −6 dB bandwidth) may be obtained in single crystal transducers with optimized electrical and acoustic design. The extra bandwidth achieved from multiple matching layer single crystal transducers can offer a wide range for division into passbands for multiple simultaneous transmit beams. Further details of the methods for manufacturing single crystal transducers may be found in U.S. Pat. No. 6,425,869, the contents of which are hereby incorporated by reference.

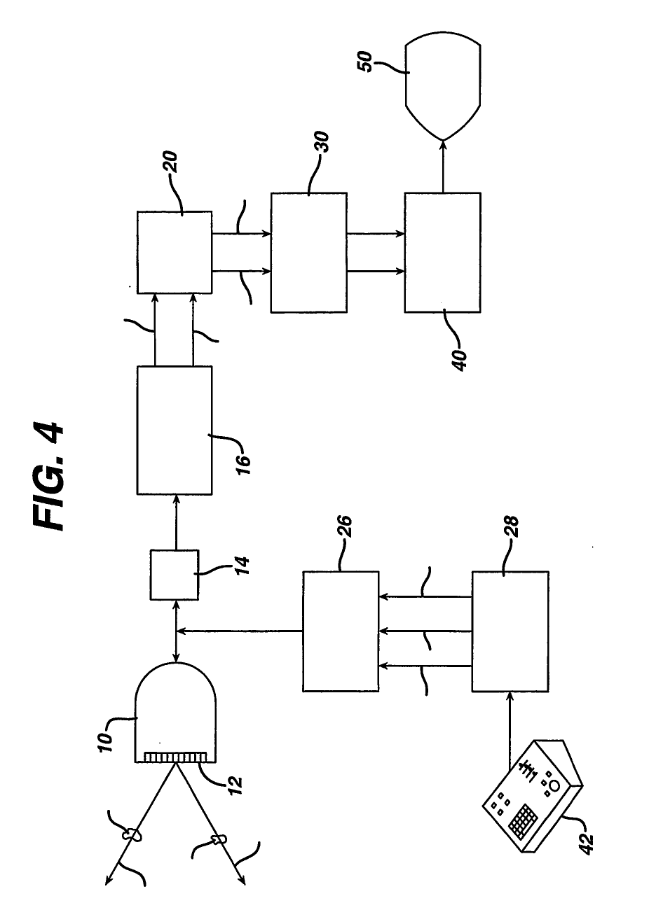

Referring to FIG. 4, an ultrasound system for operating a multiple beam transducer probe 10 in accordance with the principles of the present invention is shown in block diagram form. The probe 10 includes a single crystal array transducer 12 fabricated as discussed above. The probe is operated to simultaneously transmit two beams A and B which are steered in different directions θ₁ and θ₂ to interrogate targets T1 and T2. The term simultaneous, as used herein, means that a beam is transmitted prior to the completion of echo reception from a previously or concurrently transmitted beam. The two beams may be transmitted using differently encoded transmit pulses which have been encoded with coding schemes such as FM chirp encoding, Golay codes, or Barker codes. The transmitted beams are transmitted under control of a transmit beamformer 26, which provides transmit pulses of the desired pulse characteristics and at the appropriate times to the elements of the array transducer 12. Certain characteristics of the transmit beams may be selected by the system operator using a user interface 42. The characteristics selected by the user are input to a transmit waveform generator 28. The transmit waveform generator 28 may calculate and form the needed transmit pulses, or may select them from a pulse waveform library, or may forward control parameters such as the bands and bandwidths of the beams (BW), the steering angles of the beams (θ), and any pulse coding used (Coding) to the transmit beamformer 26 which will use the parameters to produce the necessary pulse waveforms. In response to the transmitted beams, echoes are received simultaneously along each beam direction. The received echo signals are converted to digital samples by an A/D converter 14 for each transducer element and coupled to respective channels of a multiline beamformer 16. In addition to the multiple lines A and B in different beam directions, each transmit beam can insonify multiple closely spaced receive lines if desired. Thus, for instance, with 4× multiline, the transmission of two beams can result in eight (2*4=8) multilines from a single transmit interval, thereby increasing the frame rate even further. The beamformer 16 produces two receive beams A′ and B′ in this example. These receive beams are filtered by matched filter 20 to compress the encoded echoes, thereby producing the desired receive beams A and B (and associated multilines of each beam, if produced by the beamformer). The received beams undergo signal processing in a signal processor 30 and image processing in an image processor 40 to produce a two or three dimensional image which is displayed on a display 50.

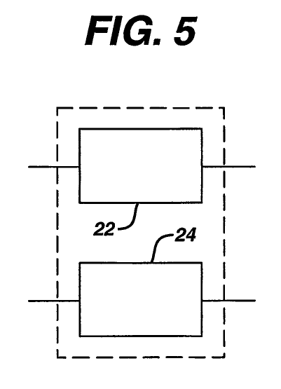

Details of the filter 20 are shown in FIG. 5. If the transmitted signals occupy completely separate bands within the passband of the transducer and within the dynamic range of the displayed signals, the echoes from unencoded transmit pulses may be separated simply by bandpass filtering, in which case the filter 20 comprises bandpass filter A (22) and bandpass filter B (24). That is, coded transmit pulses are not needed, as the echoes are in completely separate passbands A and B. However, in many applications the designer will desire as broad a bandwidth as possible to maximize axial resolution, and the passbands of the different beams will overlap. In such a case, the signal component from the first transmit beam that overlaps in frequency with another transmit beam will give rise to cross-talk in the received lines formed from the second transmit beam. Cross-talk manifests itself as ghosting artifacts or clutter in the receive lines. In this situation, where band pass filtering alone is not sufficient to separate the frequency contents of each transmit beam, coded transmit pulses are preferred and the output signals from the beamformer 16 are separated using matched filters 22 and 24. Bandpass filtering alone would produce beam A with some cross-talk “b” from beam B, and also would produce beam B with some cross-talk “a” from beam A, as shown in FIG. 5. The received echo signals are thus processed by matched filter A (22) and matched filter B (24) to remove much of the cross-talk from each A and B signal.

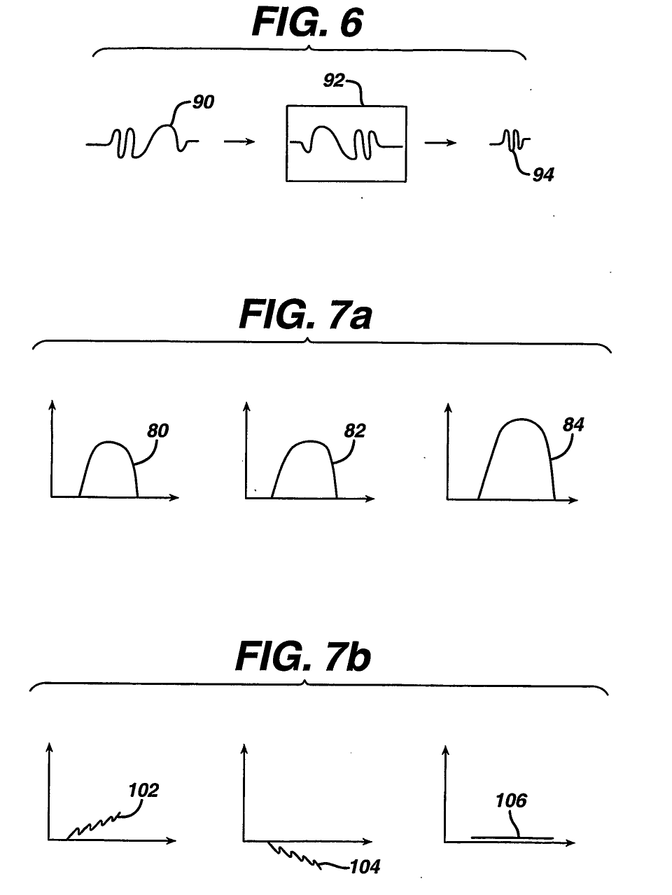

As used herein the term “matched filter” refers to a filter which, for a given signal X, has an impulse response which is the time-reversal of signal X. An example of a matched filter 92 is shown in FIG. 6. In this example the coded receive signal has a waveform in the time domain as illustrated by waveform 90. A matched filter for such a signal has an impulse response which is the time reversal of this signal, as illustrated by the waveform shown in the box 92. When the waveform 90 is processed by a filter of this characteristic, a compressed, unencoded pulse 94 is produced.

Typical amplitude and phase characteristics of a matched filter system are shown in FIGS. 7a and 7b. The first response characteristic 80 in FIG. 7a is the amplitude response characteristic of a coded receive signal. A matched filter will have a matching amplitude response 82. As a result the filter output signal will exhibit an amplitude response characteristic 84. Since the filter is matched to the signal, all characteristics have a bandwidth extending from a to b.

The signal will also exhibit a phase response 102 as illustrated in FIG. 7b. The matched filter will exhibit a complementary phase response 104. As a result the matched filter output signal will exhibit a linear phase response 106.

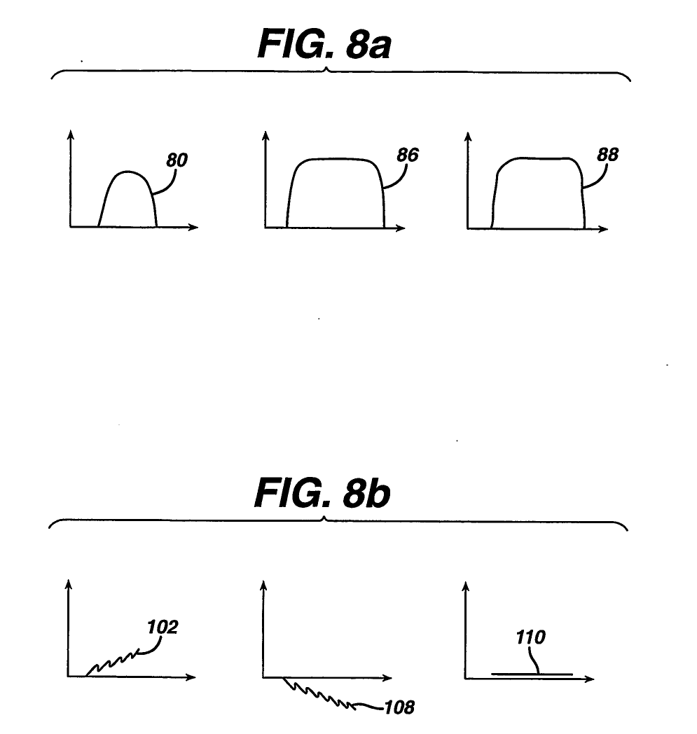

In some cases it may be desirable to enhance the axial resolution of the filtered output signal by trading off the signal-to-noise ratio for improved bandwidth. In such cases a mismatched filter may be used as illustrated by the response characteristics of FIGS. 8a and 8b. The received signal again has an amplitude response characteristic 80 which extends between frequencies a and b as shown in FIG. 8a. The mismatched filter will have a broader response characteristic 86 which is seen to extend between frequencies a′ and b′. As a result the amplitude response 88 of the mismatched filter output signal will extend between frequencies a′ and b′. The coded receive signal will exhibit a phase response 102 as shown in FIG. 8b. The mismatched filter will exhibit a closely complementary phase response characteristic 108. As a result the filter output signal will exhibit a substantially linear phase response 110 across the mismatched filter bandwidth. Due to the extended bandwidth of the mismatched filter the received signals will have a broader bandwidth providing improved axial resolution but at the expense of a reduced signal-to-noise ratio. The passbands of the matched and mismatched filters can be time-variable if desired to follow the declining frequencies of echo signals received from greater depths during echo reception.

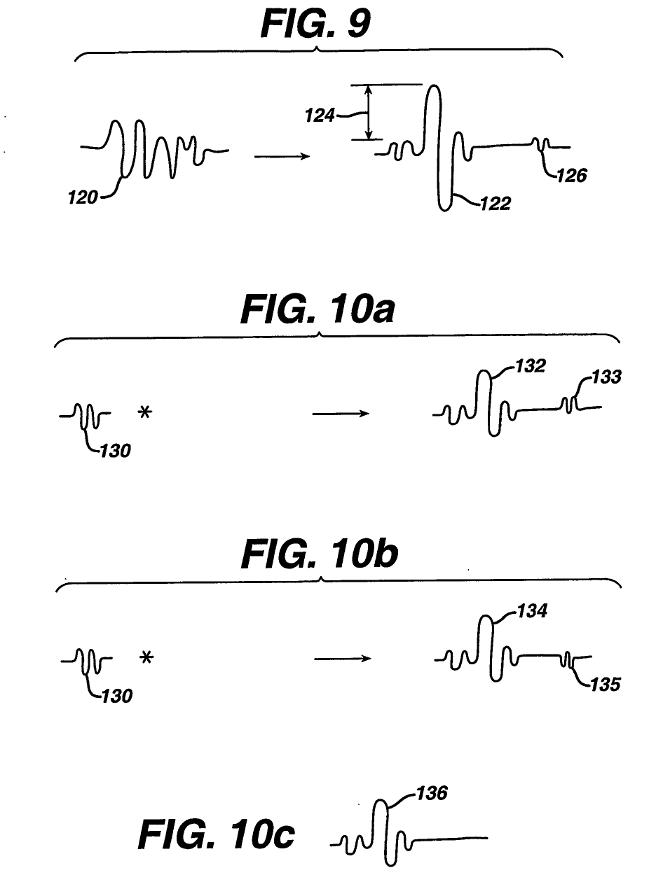

A coding scheme which provides an enhanced ratio of the main echo lobe to sidelobes is a Barker code. FIG. 9 illustrates a received echo 120 from a coded transmit pulse such as a Barker coded pulse. After matched filtering the compressed echo 122 will exhibit an enhanced ratio of the main to side lobes as indicated by arrow 124. However Barker coded pulses remain susceptible to remaining range sidelobes as shown at 126 in the filtered output signal 122. If these artifacts are a problem they may be reduced by the use of Golay coded transmit pulses. Golay codes are chosen paired complementary pseudo-random codes which exhibit the property that when the autocorrelation functions of two associated codes are added, the range sidelobes cancel (M J E Golay, “Complementary Series,” IRE Trans. on Info. Theory, Vol. IT-7, No. 4, pp. 82-87, April, 1961.) For example, FIG. 10a illustrates a first transmit pulse 130 which is encoded by a first Golay code #1. The coded pulse is transmitted and an echo received which, after decoding, exhibits a main lobe 132 and a sidelobe 133. A second transmit pulse 130 of the same form as the first pulse is encoded by a second Golay code #2 and transmitted. After filtering the received echo will exhibit a main lobe 134 and a sidelobe 135. As a result of the complementary coding the range sidelobes 133 and 135 are the complements of each other such that, when combined, they cancel, resulting in a final received signal 136 from the two coded transmissions. The final received signal is seen to be free of the canceled artifact. Golay codes, however, will generally not exhibit as favorable a main-to-side lobe ratio as will Barker codes, so the choice of coding schemes will be chosen by the designer in consideration of the most desirable attribute needed.

In a constructed embodiment of the present invention, the frequency separation provided by the wide bandwidth transducer can be expected to provide cross-talk reduction in the simultaneously received beams on the order of 10-15 dB. The use of a coding scheme for the transmitted pulses can provide another 10-12 dB of cross-talk reduction. Beamforming, which steers the spatially separated transmit and received beams, can be expected to provide another 10-15 dB of cross-talk reduction. As a result the ghosting of artifacts from one beam into another can be reduced by up to 30-42 dB by employing all three cross-talk reduction techniques while still affording good axial resolution in the simultaneously transmitted and received beams.

It will be appreciated that, while simultaneously transmitted beams are often not preferred in two dimensional imaging, three dimensional imaging applications will benefit from simultaneously transmitted beams, as such a transmit scheme can reduce the volume acquisition time and thereby improve the volume frame rate of display.

1. An ultrasonic imaging system comprising:
a probe including a single crystal transducer array exhibiting a transducer band;
a transmit beamformer coupled to elements of the transducer array and controlled to cause the probe to transmit two or more beams during the same transmit interval in different beam directions, wherein each beam occupies a substantially different bandwidths of the transducer band;
a receive beamformer coupled to process two or more receive beams in response to the transmitted beams during the same receive interval, the receive beams exhibiting steering directions corresponding to those of the transmitted beams;
a filter coupled to the beamformer which acts to filter the receive beams;
a signal processor coupled to the filter;
an image processor coupled to the signal processor; and
a display coupled to the image processor which displays an image formed from components of the receive beams.

2. The ultrasonic imaging system of claim 1, wherein the transmit beamformer further comprises a pulse encoder which acts to cause the probe to transmit differently coded transmit pulses in the different beam directions.

3. The ultrasonic imaging system of claim 2, wherein the pulse encoder comprises one of a chirp pulse encoder, a Barker code encoder, or a Golay code encoder.

4. The ultrasonic imaging system of claim 1, wherein the filter comprises bandpass filters exhibiting passbands corresponding to the different bandwidths.

5. The ultrasonic imaging system of claim 1, wherein the filter comprises two or more matched filters matched to the characteristics of the transmitted beams.

6. The ultrasonic imaging system of claim 2, wherein the filter comprises two or more matched filters matched to the characteristics of the coded transmit pulses.

7. The ultrasonic imaging system of claim 5, wherein the matched filters exhibit passbands respectively matched to the bandwidths of the anticipated received signals, and exhibit phase response characteristics which are the respective complements of the phase characteristics of the anticipated received signals.

8. The ultrasonic imaging system of claim 1, wherein the filter comprises two or more mismatched filters exhibiting characteristics chosen in considerations of the characteristics of the anticipated received signals.

9. The ultrasonic imaging system of claim 1, wherein the bandwidths of the beams are substantially non-overlapping in frequency.

10. The ultrasonic imaging system of claim 9, wherein the filter comprises a bandpass filter.

11. The ultrasonic imaging system of claim 1, wherein the bandwidths of the beams are fractionally overlapping in frequency.

12. The ultrasonic imaging system of claim 11, wherein the transmit beamformer uses differently coded pulses to transmit the beams, and wherein the filter comprises a matched filter matched to the coding of the beams.

13. The ultrasonic imaging system of claim 1, wherein the beamformer comprises a multiline beamformer.

14. The ultrasonic imaging system of claim 13, wherein the multiline beamformer acts to produce two or more beams substantially aligned with each of the steering directions of the transmitted beams.
